(19) United States
(12) Patent Application Publication
(10) Pub. No.: US 2009/0304599 A1
(43) Pub. Date: Dec. 10, 2009

(54) Protein Nanoparticles and the Use of the Same

(75) Inventors: Makiko Aimi, Kanagawa (JP); Ryoichi Nemori, Kanagawa (JP); Masayoshi Kojima, Kanagawa (JP); Madoks Kishima, Kanagawa (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM CORPORATION, Minato-ku, Tokyo (JP)

(21) Appl. No.: 12/097,560

(22) PCT Filed: Dec. 20, 2006
(86) PCT No.: PCT/JP2006/325987

§ 371 Date: Jun. 16, 2008

(30) Foreign Application Priority Data
Dec. 20, 2005 (JP) 2005-366103

(51) Int. Cl. A61K 49/18; A61K 38/43; A61K 9/14; A61K 33/26; A61P 43/00

(52) U.S. Cl. 424/9.32; 424/94.5; 424/94.1; 424/489; 424/646; 977/773

(57) ABSTRACT

An object of the present invention is to provide nanoparticles that can easily be delivered to a small site such as a capillary and can be produced using highly biocompatible and safe material. The present invention provides a nanoparticle which contains at least one pharmaceutically active component, a magnetically responsive particle, and a protein.

### TECHNICAL FIELD

The present invention relates to a protein nanoparticle which comprises a magnetically responsive particle and a pharmaceutically active component, and use thereof.

### BACKGROUND ART

Microparticle materials have been expected to be widely used in biotechnology. Particularly recently, the applications of nanoparticle materials that have been generated as a result of the progress in nanotechnology to biotechnology and medicine have been actively discussed. Thus, many study results have been reported.

Among the microparticle materials, magnetic microparticle materials have been widely used in the field of biotechnology. For instance, magnetic microparticles having substances such as antibodies immobilized thereon have been used for immunodiagnosis. In addition, magnetic microparticles having DNAs immobilized on the surfaces thereof have been widely used in the field of genetic engineering for the purposes of separation of mRNA and single-stranded DNA, separation of DNA-binding proteins, and the like. Moreover, magnetic nanoparticles are very useful for protein interaction analysis that is one of the important means in proteome analysis.

Also, in the field of medical diagnosis, magnetic nanoparticles have been found effective when used in the form of a contrast medium for MRI diagnosis and used in cancer thermotherapy. Cancer cells are killed by heating at 42.5° C. or more (Dewey, W. C., Radiology, 123, 463-474 (1977)).

In the current thermotherapy, normal tissue and tumor tissue are heated together without distinction therebetween. Thus, based on consideration of burdens on patients, the temperature is controlled at approximately 42.5° C., at which there are few effects on normal tissue. However, it is obvious that cancer cells are likely to be killed as the heating temperature rises. Therefore, if it is possible to heat tumor tissue in a specific manner without heating normal tissue, it becomes theoretically possible to kill any type of cancer cell. Accordingly, induction heating-type thermotherapy has been developed, upon which magnetite (Fe₃O₄) in the form of magnetic nanoparticles is used for heating elements. Hitherto, regression of various types of carcinoma (brain tumor, skin cancer, tongue cancer, breast cancer, hepatocellular carcinoma, and osteosarcoma) has been achieved in various types of animal species (mice, rats, hamsters, and rabbits) (e.g., Kobayashi, T., Jpn. J. Cancer Res., 89, 463-469 (1998); and Kobayashi, T., Melanoma Res., 13, 129-135 (2003)).

Since magnetic nanoparticles have small (nano-scale) particle sizes, such particles are highly excellent in terms of dispersibility in an aqueous solution and a molecule-recognizing properties as compared with conventionally used micron-size magnetic particles or latex beads. Accordingly, it is expected that the improved sensitivity and the shortening of measurement time will be achieved to a great extent only by substituting conventionally used magnetic microparticles, latex carriers, and the like with magnetic nanoparticles.

Meanwhile, in the field of drug delivery system (DDS), the usefulness of nanoparticles has been expected since early on. Nanoparticles are very promising as carriers of pharmaceuticals and genes. In order to improve treatment efficiency with the use of anticancer agents, it is necessary to perform targeting techniques whereby pharmaceuticals are allowed to act exclusively on cancer cells or cancer lesions. With the use of magnetic properties, noninvasive in vivo direction and localization of a substance become possible.

Kato et al. have developed ethylcellulose microcapsules (hereafter referred to as FM-MMC-mc) having a diameter of 250 μm which encapsulates mitomycin C and ferrite magnetic powders. During a therapeutic trial involving VX tumors that were implanted in the upper legs of domestic rabbits, obvious antitumor effects were observed in a group subjected to magnetic direction of FM-MMC-mc as compared with a group to which MMC in a general form had been administered. This was because magnetism caused MMCs in capsules that accumulated in small arteries of tumors to be released into neighboring tumor tissue over a long period of time. Thus, this fact strongly suggests that a targeting therapy can be carried out whereby strong effects that could not be obtained by conventional methods can be provided (e.g., Kato, Tetsuro, “Increased efficacy of an anticancer agent in microcapsules due to magnetic field direction,” Japanese Journal of Cancer and Chemotherapy, 8(5), 698-706, 1981).

The size of the aforementioned FM-MMC-mc is as large as 250 μm, so that it cannot be delivered to a small site such as a capillary. In addition, since ethylcellulose is a synthetic polymer, it is problematic in terms of safety.

In addition, JP Patent Publication (Kohyo) No. 2001-502721 A teaches a drug targeting system which employs nanoparticles made of polymer material. JP Patent Publication (Kohyo) No. 2005-500304 A teaches spherical protein particles. These particles do not contain magnetically responsive particles. Thus, it is impossible to direct nanoparticles to lesions via magnetic force. JP Patent Publication (Kokai) No. 2000-256015 A teaches a metal oxide complex wherein metal oxide particles having particle sizes of 5 to 200 nm are dispersed in at least the surface layer of a gel product. However, such particles do not have the functions of DDS.

Crosslinking of proteins is generally chemical crosslinking. In accordance with known methods of such chemical crosslinking, the addition of the above crosslinking agent such as glutaraldehyde, UV irradiation using monomers having photoactive groups, localized generation of radicals due to pulse irradiation, and the like are carried out. Meanwhile, in the case of a method wherein properties of biopolymers are utilized, transglutaminase is used to catalyze a translocation reaction of acyl of glutamine residues, resulting in intermolecular and intramolecular crosslinking formation (e.g., JP Patent Publication (Kokai) No. 64-27471 A (1989)). However, in general, such method is carried out in bulk or moistened biopolymers, and crosslinking formation in protein nanoparticles has not been known. Moreover, a crosslinking reaction in nanoparticles dispersed in an organic solvent is not known.

### DISCLOSURE OF THE INVENTION

It is an object of the present invention to solve the problems of the aforementioned conventional techniques. That is, it is an object of the present invention to provide nanoparticles that can easily be delivered to a small site such as a capillary and can be produced using highly biocompatible and safe material.

As a result of intensive studies to attain above objects, the inventors of the present invention have found that protein nanoparticles containing magnetically responsive particles and medically active substances can be produced by mixing an aqueous dispersion of magnetically responsive particles, a protein, an enzyme having a crosslinking action, and a medically active substance, followed by agitation. The present invention has been completed based on these findings.

That is, the present invention provides a nanoparticle which contains at least one pharmaceutically-active component, a magnetically responsive particle, and a protein.

Preferably, the protein is crosslinked during or after nanoparticle formation.

Preferably, a crosslinking treatment is carried out by adding a crosslinking agent in an amount of 0.1% to 100% by weight relative to the weight of the protein.

Preferably, the crosslinking agent is an inorganic or organic crosslinking agent.

Preferably, the crosslinking agent is an enzyme, and further preferably the crosslinking agent is transglutaminase.

Preferably, disulfide bonds in protein molecules are reduced, and crosslinking takes place via the reformation of disulfide bond after particle formation.

Preferably, the average particle size is 10 to 1000 nm.

Preferably, the pharmaceutically active component is an anticancer agent, an antiallergic agent, an antioxidant, an antithrombotic agent, an antiinflammatory agent, an immunosuppressing agent, or a nucleic acid drug.

Preferably, the magnetically responsive particle is an iron oxide nanoparticle.

Preferably, the nanoparticle of the present invention contains a magnetically responsive particle in an amount of 0.1% to 100% by weight of the weight of the protein.

Preferably, the protein is collagen, gelatin, albumin, globulin, casein, transferrin, fibroin, fibrin, laminin, fibronectin, or vitronectin.

Preferably, the protein is one which is derived from bovine, swine or fish, or a recombinant protein.

Further preferably, the protein is acid-treated gelatin.

Preferably, a phospholipid is added in an amount of 0.1% to 100% by weight relative to the weight of the protein.

Preferably, cationic or anionic polysaccharide is added in an amount of 0.1% to 100% by weight relative to the weight of the protein.

Preferably, cationic or anionic protein is added in an amount of 0.1% to 100% by weight relative to the weight of the protein.

Another aspect of the present invention provides an MRI contrast medium which contains the nanoparticle of the present invention.

Further another aspect of the present invention provides a drug delivery agent which contains the nanoparticle of the present invention.

Further another aspect of the present invention provides a method of directing a nanoparticle to a lesion site, which comprises administering in vivo the nanoparticle of the present invention and directing the nanoparticle to a lesion site via magnetic force.

Further another aspect of the present invention provides a method of directing a nanoparticle to a lesion site, which comprises administering in vivo the nanoparticle of the present invention, directing the nanoparticle to a lesion site via magnetic force, and confirming the nanoparticle which has been directed to the lesion by MRI contrast test.

Further another aspect of the present invention provides a drug delivery method which comprises administering in vivo the nanoparticle of the present invention, directing the nanoparticle to a lesion via magnetic force, heating the nanoparticle by irradiation with high-frequency waves, and releasing a pharmaceutically active component encapsulated in the nanoparticle.

Further another aspect of the present invention provides a drug delivery method, which comprises administering in vivo the nanoparticle of the present invention, directing the nanoparticle to a lesion via magnetic force, confirming the nanoparticle which has been directed to the lesion by MRI contrast test, heating the nanoparticle by irradiation with high-frequency waves, and releasing a pharmaceutically active component encapsulated in the nanoparticle.

### BRIEF DESCRIPTION OF THE DRAWINGS

FIG. 1 shows images of the iron oxide nanoparticle of the present invention. In the image below, the center black spot denotes iron oxide, and the gray portions around the spot denote gelatin nanoparticles (approximately 150 nm).

FIG. 2 shows a result indicating that the iron oxide nanoparticle of the present invention was attracted by a magnet.

FIG. 3 shows a photograph of BAE cells immediately after addition of nanoparticle dispersion liquid.

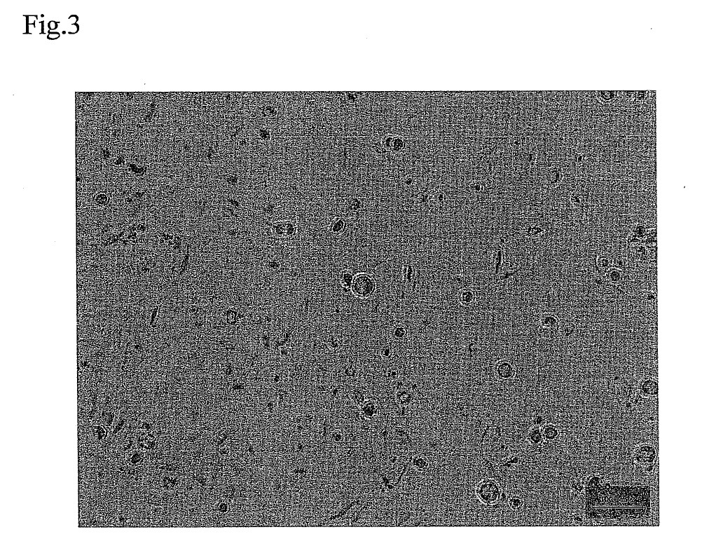

FIG. 4 shows a photograph of BAE cells after 72 hour culture.

FIG. 5 shows a photograph (enlarged) of BAE cells after 72 hour culture

### BEST MODE FOR CARRYING OUT THE INVENTION

Hereafter, the embodiments of the present invention will be described below in greater detail.

The nanoparticle of the present invention is characterized in that it contain at least one pharmaceutically active component, a magnetically responsive particles, and a protein. The protein contained in the nanoparticle of the present invention may be or may not be subjected to a crosslinking treatment. However, preferably, the protein is subjected to a crosslinking treatment. Further preferably, the protein is subjected to a crosslinking treatment during or after nanoparticle formation. The protein may be subjected to a crosslinking treatment with the use of a crosslinking agent. Alternatively, disulfide bonds in the protein molecules are reduced, and crosslinking takes place via reformation of disulfide bond after particle formation. The crosslinking treatment in the present invention may be carried out by a single method of crosslinking or by a combination of two or more methods of crosslinking.

When a crosslinking agent is used, preferably, a crosslinking treatment can be carried out by adding a crosslinking agent in an amount of 0.1% to 100% by weight relative to the weight of the protein.

As a crosslinking agent, an inorganic or organic crosslinking agent, an enzyme, or the like can be used. Examples of an inorganic or organic crosslinking agent include, but are not limited to, chromium salts (e.g., chromium alum and chromium acetate); calcium salts (e.g., calcium chloride and calcium hydroxide); aluminium salts (e.g., aluminium chloride and aluminum hydroxide); carbodiimides (e.g., EDC, WSC, N-hydroxy-5-norbornene-2,3-dicarboximide (HONB), N-hydroxysuccinimide(HOSu), and dicyclohexylcarbodiimide (DCC)); N-hydroxysuccimide; and phosphorus oxychloride. The enzyme is not particularly limited as long as it has a crosslinking action on protein. Preferably, transglutaminase can be used. Specifically, proteins subjected to enzymatic crosslinking using transglutaminase are not particularly limited as long as they have lysine residues and glutamine residues. Preferred examples thereof include acid-treated gelatin, collagen, and albumin.

The transglutaminase may be one derived from mammals or microorganisms. Specific examples thereof include Activa series (Ajinomoto Co., Inc.) and mammalian-derived transglutaminases that are commercially available as reagents such as guinea pig liver-derived transglutaminase, goat transglutaminase, and rabbit-derived transglutaminase, which are produced by Oriental Yeast Co., Ltd., Upstate USA Inc., Biodesign International, and the like. The transglutaminase may be human derived-recombinant transglutaminase.

The above crosslinking agent may be used alone or in combination of two or more.

In the present invention, a reducing agent is used when disulfide bonds in the protein molecule are reduced and crosslinking takes place via reformation of disulfide bond after particle formation. Specific examples of the reducing agent include, but are not limited to, the following compounds: thioglycolates such as dithiothreitol, thioglycolic acid, and ammonium thioglycolate; cysteinates such as cysteine and cysteine hydrochloride; cysteine derivatives such as N-acetylcysteine; monoglyceride thioglycolate; cysteamine; thiolactic acid; sulfite; bisulfite; and mercaptoethanol.

The average particle size of the nanoparticle of the present invention is generally 1 to 1000 nm, preferably 10 to 1000 nm, more preferably 50 to 500 nm, and particularly preferably 100 to 500 nm. Since the nanoparticle of the present invention has nano-order size as described above, it can be delivered to a small site such as a capillary.

The type of the pharmaceutically active component contained in the nanoparticle of the present invention is not particularly limited. Preferably, the pharmaceutically active component is an anticancer agent, antiallergic agent, antioxidant, antithrombotic agent, an antiinflammatory agent, an immunosuppressing agent, or a nucleic acid drug, and particularly preferably an anticancer agent.

Specific examples of an anticancer agent that can be used in the present invention include, but are not limited to, pyrimidine fluoride antimetabolites (e.g., 5-fluorouracil (5FU), tegafur, doxifluridine, and capecitabine), antibiotics (e.g., mitomycin (MMC) and Adriacin (DXR)), purine antimetabolites (e.g., folic acid antimetabolites such as methotrexate, and mercaptopurine), vitamin A active metabolites (e.g., antimetabolites such as hydroxy carbamide, tretinoin, and tamibarotene), molecular targeting agents (e.g., Herceptin and imatinib mesylate), platinum drugs (e.g., Briplatin and Randa (CDDP), Paraplatin (CBDC), Elplat (Oxa), and Aqupla), plant alkaloids (e.g., Topotecin, Campto (CPT), Taxol (PTX), Taxotere (DTX), and Etoposide), alkylating agents (e.g., Busulfan, cyclophosphamide, and Ifomide), antiandrogens (e.g., bicalutamide and flutamide), female hormones (e.g., Fosfestrol, chlormadinone acetate, and estramustine phosphate), LH-RH agonists (e.g., Leuplin and Zoladex), antiestrogens (e.g., tamoxifen citrate and toremifene citrate), aromatase inhibitors (e.g., fadrozole hydrochloride, anastrozole, and Exemestane), progestins (e.g., medroxyprogesterone acetate), and BCG.

Specific examples of an antiallergic agent that can be used in the present invention include, but are not limited to, mediator release suppressing agents such as sodium cromoglicate or tranilast, histamine H1-antagonists such as ketotifen fumarate or azelastine hydrochloride, thromboxane inhibitors such as ozagrel hydrochloride, leukotriene antagonists such as pranlucast, and suplatast tosylate.

Specific examples of an antioxidant that can be used in the present invention include, but are not limited to, vitamin C and its derivative, vitamin E, kinetin, α-lipoic acid, coenzyme Q10, polyphenol, SOD and phytic acid.

Specific examples of an antithrombotic agent that can be used in the present invention include, but are not limited to, aspirin, ticlopidine hydrochloride, cilostazol and warfarin potassium.

Specific examples of an antiinflammatory agent that can be used in the present invention include, but are not limited to, a compound which is selected from azulene, allantoin, lysozyme chloride, guaiazulene, diphenhydramine hydrochloride, hydrocortisone acetate, prednisolone, glycyrrhizinic acid, glycyrrhetinic acid, glutathione, saponin, methyl salicylate, mefenamic acid, phenylbutazone, indometacin, ibuprofen and ketoprofen, and its derivative and its salt; and a plant extract which is selected from Scutellariae Radix extract, Artemisia capillaris Thunb. Extract, Platycodon grandiflorum extract, Armeniacae Semen extract, Common gardenia extract, Sasa veitchii extract, Gentiana lutea extract, Comfrey extract, white birch extract, Malva extract, Persicae Semen extract, peach blade extract, and loquat blade extract.

Specific examples of an immunosuppressing agent that can be used in the present invention include, but are not limited to, rapamycin, tacrolimus, cyclosporin, prednisolone, methylprednisolone, mycophenolate mofetil, azathioprine and mizoribine.

Specific examples of a nucleic acid drug that can be used in the present invention include, but are not limited to, antisense nucleic acid, ribozyme, siRNA, aptamer and decoy nucleic acid.

The pharmaceutically active component may be added upon or after nanoparticle formation.

Preferably in the present invention, a substance having selective affinity to cancer cells can be added to the nanoparticle. Particularly preferably, an antibody or folic acid can be added. An example of an antibody having selective affinity to cancer cells that can be used is an antibody which recognizes a cancer antigen. Preferably, an antibody which recognizes a free antigen can be used. Specific examples of such cancer antigen include an epidermal growth factor receptor (EGFR), an estrogen receptor (ER), and a progesterone receptor (PgR).

A person skilled in the art can readily obtain the above antibody having selective affinity to cancer cells. For instance, commercially available antibodies may be used. Alternatively, antibodies that are used in the present invention can be produced according to need based on known methods for producing antibodies using the above antigens or partial peptides thereof as an immunogen. In addition, the antibody used may be a monoclonal or polyclonal antibody.

The antibody described above can react with an amino group or a carboxyl group of the protein contained in the nanoparticle of the present invention. Thus, the antibody can bind to the nanoparticle of the present invention via peptide bond formation or the like as a result of an amidation reaction.

An amidation reaction is carried out via condensation of a carboxyl group or derivative group thereof (e.g., ester, acid anhydride, and acid halide) and an amino group. When acid anhydride or acid halide is used, it is preferable that bases coexist with it. When an ester such as methyl ester or ethyl ester of carboxylic acid is used, it is desirable that heating or pressure reduction be carried out such that generated alcohol can be removed. When a carboxyl group is directly subjected to amidation, it is possible to allow the following substances that promote amidation reaction to coexist with or previously react with the carboxyl group: amidation reagents such as DCC, Morpho-CDI, and WSC; condensation additives such as HBT; and active esterifying agents such as N-hydroxyphthalimide, p-nitrophenyl-trifluoroacetate, and 2,4,5-trichlorophenol. In addition, upon an amidation reaction, it is desirable that either an amino group or a carboxyl group of the affinity molecules to be bound via amidation be protected with adequate protecting groups in accordance with conventional methods, followed by deprotection after the reaction.

The nanoparticle that has bound to the antibody having selective affinity to cancer cells via an amidation reaction can be washed and purified by conventional techniques such as gel filtration, and then can be dispersed in water and/or a hydrophilic solvent (preferably, methanol, ethanol, isopropanol, 2-ethoxyethanol, or the like). Thereafter, the nanoparticle can be used.

Any types of a magnetically responsive particle can be used in the present invention, as long as it is harmless to human bodies and absorb electromagnetic waves so as to generate heat. In particular, it is preferable to use a magnetically responsive particle that generate heat by absorbing electromagnetic waves having frequencies at which electromagnetic waves are unlikely to be absorbed by human bodies. Preferably, the magnetically responsive particle is ferroplatinum, iron oxide, or ferrite (Fe, M)₃O₄, and particularly preferably iron oxide nanoparticles. Herein, specific examples of iron oxide include Fe₃O₄ (magnetite), γ-Fe₂O₃ (maghemite), and intermediates and mixtures thereof. In addition, the particle may have a core-shell structure where the composition of the surface differs from that of the inside. In the above formula, “M” denotes a metal ion that can form magnetic metallic oxide when used together with the iron ion. A typical example thereof is selected from among transition metals. The most preferred examples thereof include Zn²⁺, Co²⁺, Mn²⁺, Cu²⁺, Ni²⁺, and Mg²⁺. The molar ratio of M to Fe is determined based on the stoichiometric composition of ferrite to be selected.

The size of the magnetically responsive particle used in the present invention is preferably 1 to 1000 nm, more preferably 1 to 500 nm, and particularly preferably 5 to 100 nm.

Preferably, the nanoparticle of the present invention can contain the magnetically responsive particle in an amount of 0.1% to 100% by weight relative to the weight of the protein.

The types of the protein used in the present invention are not particularly limited; however, it is preferable to use a protein having a molecular weight of 10,000 to 1,000,000. The origin of the protein is not particularly limited; however, the protein is one which is derived from bovine, swine or fish, or a recombinant protein. It is preferable to use human-derived proteins. For example, the proteins described in EP 1014176A2 and U.S. Pat. No. 6,992,172 can be used.

Examples of the protein that can be used include collagen, gelatin or acid-treated gelatin, albumin, globulin, casein, transferring, fibroin, fibrin, laminin, fibronectin, and vitronectin.

The protein nanoparticle of the present invention can be produced in accordance with the method described in JP Patent Publication (Kokai) No. 6-79168 A (1994) or the method described in “Journal of Microencapsulation,” C. Coester, 2000, vol. 17, pp. 187-193. Preferably, the crosslinking agent described above can be used instead of glutaraldehyde.

Specific examples of phospholipids used in the present invention include, but are not limited to, the following compounds: phosphatidyl choline (lecithin), phosphatidylethanolamine, phosphatidylserine, phosphatidylinositol, phosphatidylglycerol, diphosphatidylglycerol, and sphingomyelin.

The anionic polysaccharide used in the present invention is a polysaccharide having acid polar group such as a carboxyl group, a sulfate group or a phosphate group. Specific examples thereof include, but are not limited to, the following compounds: chondroitin sulfate, dextran sulfate, carboxymethyldextran, alginic acid, pectin, carrageenan, fucoidan, agaropectin, porphyran, karaya gum, gellan gum, xanthan gum, and hyaluronic acid.

The cationic polysaccharide used in the present invention is a polysaccharide having a basic polar group such as an amino group. Specific examples thereof include, but are not limited to, those containing galactosamines or glucosamines as the monosaccharide unit, such as chitin and chitosan.

The anionic protein used in the present invention is a protein or lipoprotein having an isoelectric point higher than physiological pH. Specific examples thereof include, but are not limited to, the following compounds: polyglutamic acid, polyaspartic acid, lysozyme, cytochrome C, ribonuclease, trypsinogen, chymotrypsinogen, and α-chymotrypsin.

The cationic protein used in the present invention is a protein or lipoprotein having an isoelectric point lower than physiological pH. Specific examples thereof include, but are not limited to, the following compounds: polylysine, polyarginine, histone, protamine, and ovalbumin.

The above nanoparticle of the present invention contains a magnetically responsive particle. Thus, it is possible to direct it to a certain site with the use of magnetic force. That is, the nanoparticle of the present invention can be administered in vivo so as to be directed to disease lesions via magnetic force. In addition, it can be confirmed by MRI contrast test that the nanoparticle has been directed to such lesions. Namely, the nanoparticle of the present invention is useful as a MRI contrast medium.

Further, after the nanoparticle of the present invention is directed to disease lesions in accordance with the above method, it is heated using high-frequency waves so that the pharmaceutically active component encapsulated in the nanoparticle can be released. That is, the nanoparticle of the present invention is useful as a drug delivery agent.

The route of administration of the nanoparticle of the present invention is not particularly limited. Preferably, the nanoparticle can be administered into blood vessels, body cavities, or lymph, by injection. Particularly preferably, the nanoparticle can be administered by intravenous injection.

The dose of the nanoparticle of the present invention can adequately be determined based on the patient's weight and the condition of the disease, for example. In general, approximately 10 μg to 100 mg/kg and preferably 20 μg to 50 mg/kg of the nanoparticle of the present invention can be administered as a single dose.

The present invention is hereafter described in greater detail with reference to the following examples, but the technical scope of the present invention is not limited thereto.

### EXAMPLE

### Example 1

Iron (III) chloride hexahydrate (10.8 g) and iron (II) chloride tetrahydrate (6.4 g) were each dissolved in 80 ml of 1 mol/l (1N) hydrochloric acid aqueous solution, and the two resulting solutions were mixed together. While the obtained solution was being agitated, 96 ml of ammonia water (28% by weight) was added thereto at a rate of 2 ml/minute. Then, the solution was heated at 80° C. for 30 minutes and cooled to room temperature. The obtained aggregate was purified with water by decantation. As a result, generation of iron oxide having a crystallite size of approximately 12 nm was confirmed by an X-ray diffraction method. The solvent was substituted with ethanol. Then, 8 ml of tetramethylammonium hydroxide (25% by weight) and 3 ml of a gelatin aqueous solution were added thereto, followed by agitation at 60° C. for 4 hours. The resulting precipitate was filtered and redispersed in water. Thus, iron oxide nanoparticle having surfaces covered with gelatin was synthesized.

Then, 0.21 ml of the above aqueous dispersion containing iron oxide nanoparticle (4.7 g/l), 20 mg of acid-treated gelatin, 10 mg of a transglutaminase formulation (Activa TG-S, Ajinomoto Co., Inc.), 0.3 mg of a pharmaceutical model having the structure shown below, and 1.79 ml of ion exchange water were mixed. The resulting solution (1 ml) was injected into 10 ml of ethanol using a microsyringe under agitation at 800 rpm at 40° C. The obtained dispersion liquid was allowed to stand for 5 hours at 55° C. Thus, cross-linked acid-treated gelatin nanoparticles were obtained.

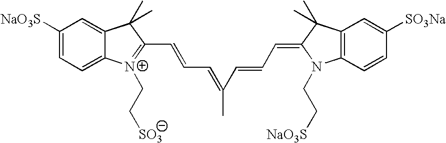

### The Pharmaceutical Model of Example 1

The average particle size of the above particles was measured using a light scattering photometer (DLS-7000, Otsuka Electronics Co., Ltd.). The average particle size was 140 nm n.

### Example 2

Iron oxide nanoparticles were synthesized in a manner similar to that used for Example 1.

0.21 ml of the above dispersion liquid containing iron oxide nanoparticles (4.7 g/l), 20 mg of acid-treated gelatin, 10 mg of a transglutaminase formulation (Activa TG-S, Ajinomoto Co., Inc.), 0.3 mg of adriamycin, and 1.79 ml of ion exchange water were mixed. The resulting solution (1 ml) was injected into 10 ml of ethanol using a microsyringe under agitation 800 rpm at 400C. The obtained dispersion liquid was allowed to stand for 5 hours at 55° C. Thus, cross-linked acid-treated gelatin nanoparticles were obtained.

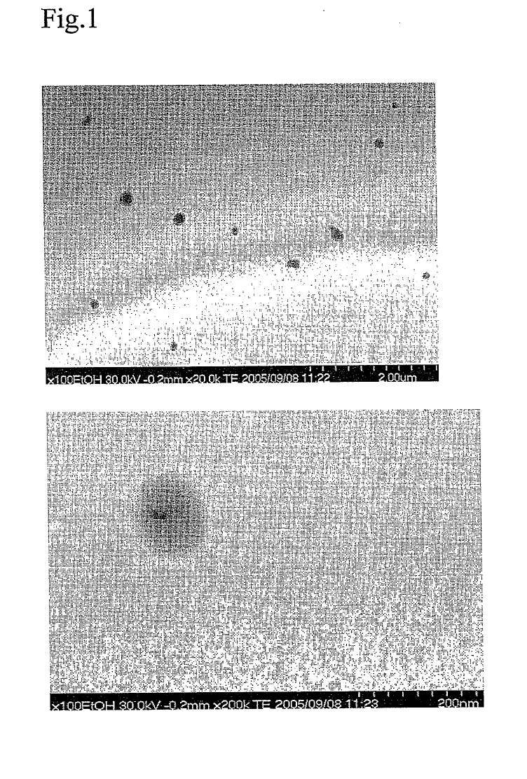

The average particle size of the above particles was measured using a light scattering photometer (DLS-7000, Otsuka Electronics Co., Ltd.). The average particle size was 160 nm. FIG. 1 shows SEM images of the particles.

### Example 3

Iron oxide nanoparticles were synthesized in a manner similar to that used for Example 1.

0.21 ml of the above dispersion liquid containing iron oxide (4.7 g/l), 20 mg of acid-treated gelatin, 10 mg of a transglutaminase formulation (Activa TG-S, Ajinomoto Co., Inc.), 0.3 mg of 5-fluorouracil, and 1.79 ml of ion exchange water were mixed. The resulting solution (1 ml) was injected into 10 ml of ethanol using a microsyringe under agitation at 800 rpm at 40° C. The obtained dispersion liquid was allowed to stand for 5 hours at 55° C. Thus, cross-linked acid-treated gelatin nanoparticles were obtained. The average particle size of the above particles was measured using a light scattering photometer (DLS-7000, Otsuka Electronics Co., Ltd.). The average particle size was 160 nm.

### Example 4

Iron oxide nanoparticles were synthesized in a manner similar to that used for Example 1.

0.21 ml of the above dispersion liquid containing iron oxide (4.7 g/l), 20 mg of aqua collagen (Chisso Corporation), 10 mg of a transglutaminase formulation (Activa TO-S, Ajinomoto Co., Inc.), 0.3 mg of adriamycin, and 1.79 ml of ion exchange water were mixed. The above solution (1 ml) was injected into 10 ml of ethanol using a microsyringe under agitation at 800 rpm at 40° C. The obtained dispersion liquid was allowed to stand for 5 hours at 55° C. Thus, cross-linked aqua collagen nanoparticles were obtained. The average particle size of the above particles was measured using a light scattering photometer (DLS-7000, Otsuka Electronics Co., Ltd.). The average particle size was 270 nm.

### Example 5

Iron oxide nanoparticles were synthesized in a manner similar to that used for Example 1.

Albumin was dissolved in a 0.5 M Tris-hydrochloride buffer (pH 8.5) containing 3 ml of 7 M guanidine hydrochloride and 10 mM EDTA. Then, 10 mg of dithiothreitol was added thereto, followed by mixing. The resultant mixture was reduced for 2 hours at room temperature, followed by purification by gel filtration. The obtained albumin solution was mixed with 0.21 ml of the dispersion liquid containing iron oxide (4.7 g/l) and 0.3 mg of adriamycin. The resulting solution (1 ml) was injected into 10 ml of ethanol in which 5 mg of calcium chloride had been dissolved, using a microsyringe under agitation at 800 rpm at 40° C. The obtained dispersion liquid was allowed to stand for 5 hours at 55° C. Thus, cross-linked albumin nanoparticles were obtained. The average particle size of the above particles was measured using a light scattering photometer (DLS-7000, Otsuka Electronics Co., Ltd.). The average particle size was 290 nm.

### Example 6

Iron oxide nanoparticles were synthesized in a manner similar to that used for Example 1.

0.21 ml of the dispersion liquid containing iron oxide (4.7 g/l), 20 mg of acid-treated gelatin, 2 mg of chondroitin sulfuric acid-C, 10 mg of transglutaminase, 0.3 mg of adriamycin, and 1.79 ml of ion exchange water were mixed. The resulting solution (1 ml) was injected into 10 ml of ethanol using a microsyringe under agitation at 800 rpm at 40° C. The obtained dispersion liquid was allowed to stand for 5 hours at 55° C. Thus, nanoparticles covered with cross-linked acid-treated gelatin were obtained.

The average particle size of the above particles was measured using a light scattering photometer (DLS-7000, Otsuka Electronics Co., Ltd.). The average particle size was 220 μm. Compared with Example 2, the encapsulation efficiency of adriamycin was increased.

### Example 7

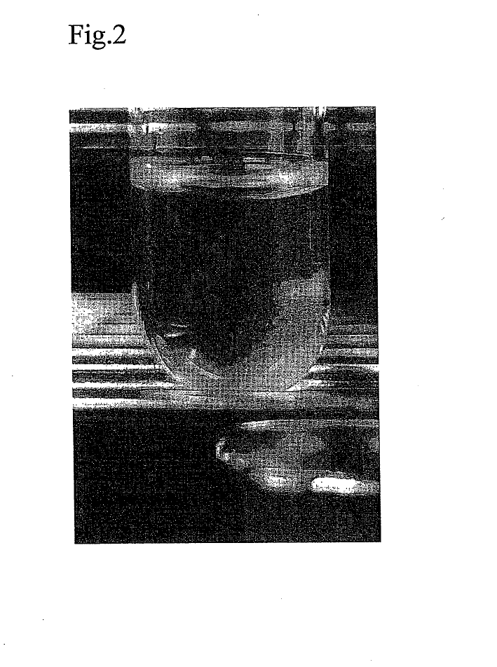

Nanoparticles (1 ml) produced in Example 2 were placed in a test tube. The bottom of the test tube was brought close to a magnet. Then, all nanoparticles were attracted by the magnet within 10 minutes (FIG. 2).

### Example 8

5 ml of saline solution was added to 11 ml of the nano particle dispersion liquid prepared in Example 6, and ethanol was distilled away by rotary evaporator. Saline solution was added so that the total volume is 10 ml.

Bovine vascular endothelial cells (BAE cells) were cultured at 1×10⁴ cells/well (96 well plate) in MEM medium supplemented with 10% fetal bovine serum and antibiotics (penicillin and streptomycin) in 5% CO₂ at 37° C.

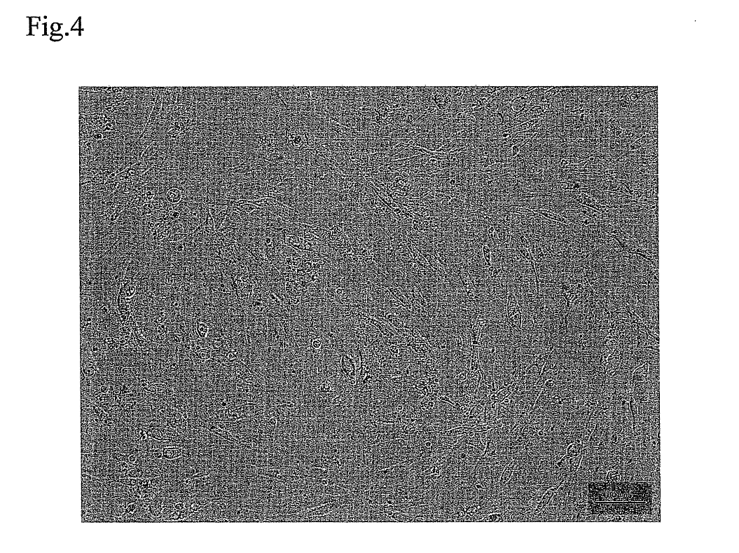

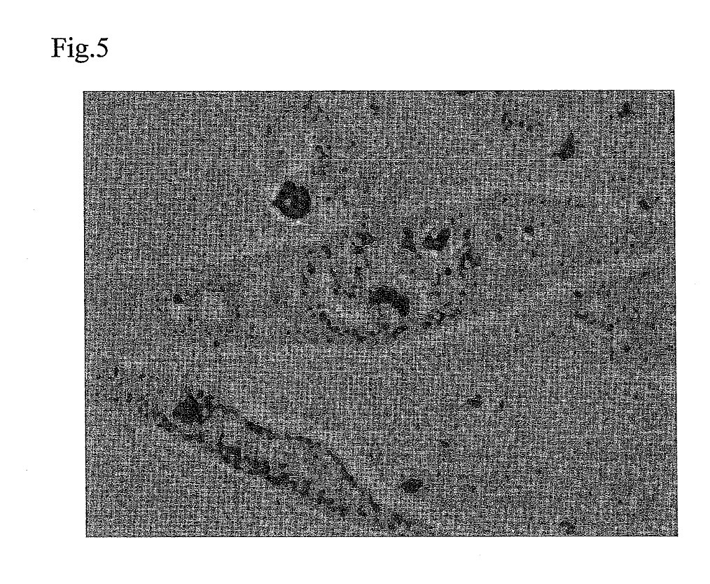

50 μl of the above dispersion liquid was added to the bovine vascular endothelial cells, and the cells were cultured 72 hours. As a result, incorporation of the nanoparticles into the cells was observed (FIGS. 4 and 5).

### INDUSTRIALLY APPLICABILITY

The nanoparticle of the present invention can easily be delivered to a small site such as a capillary. In addition, a surfactant and a synthetic polymer is not used for the nanoparticle of the present invention, and there are no remaining synthetic crosslinking agents. The nanoparticle of the present invention comprising highly biocompatible proteins is extremely safe. The nanoparticle of the present invention contains a magnetic nanoparticle and a pharmaceutical in combination. Thus, a contrast test, thermotherapy and DDS can be simultaneously carried out.

1. A nanoparticle which contains at least one pharmaceutically active component, a magnetically responsive particle, and a protein, wherein the protein is crosslinked during or after nanoparticle formation.

2. (canceled)

3. The nanoparticle of claim 1, wherein the crosslinking treatment is carried out by adding a crosslinking agent in an amount of 0.1% to 100% by weight relative to the weight of the protein.

4. The nanoparticle of claim 3, wherein the crosslinking agent is an inorganic or organic crosslinking agent.

5. The nanoparticle of claim 3, wherein the crosslinking agent is an enzyme.

6. The nanoparticle of claim 5, wherein the crosslinking agent is transglutaminase.

7. The nanoparticle of claim 1, wherein disulfide bonds in protein molecules are reduced, and crosslinking takes place via the reformation of disulfide bond after particle formation.

8. The nanoparticle of claim 1, wherein the average particle size is 10 to 1000 nm.

9. The nanoparticle of claim 1, wherein the pharmaceutically active component is an anticancer agent, an antiallergic agent, an antioxidant, an antithrombotic agent, an antiinflammatory agent, an immunosuppressing agent, or a nucleic acid drug.

10. The nanoparticle of claim 1, wherein the magnetically responsive particle is an iron oxide nanoparticle.

11. The nanoparticle of claim 1, which contains a magnetically responsive particle in an amount of 0.1% to 100% by weight of the weight of the protein.

12. The nanoparticle of claim 1, wherein the protein is collagen, gelatin, albumin, globulin, casein, transferrin, fibroin, fibrin, laminin, fibronectin, or vitronectin.

13. The nanoparticle of claim 1, wherein the protein is one which is derived from bovine, swine or fish, or a recombinant protein.

14. The nanoparticle of claim 1, wherein the protein is acid-treated gelatin.

15. The nanoparticle of claim 1, wherein a phospholipid is added in an amount of 0.1% to 100% by weight relative to the weight of the protein.

16. The nanoparticle of claim 1, wherein cationic or anionic polysaccharide is added in an amount of 0.1% to 100% by weight relative to the weight of the protein.

17. The nanoparticle of claim 1, wherein cationic or anionic protein is added in an amount of 0.1% to 100% by weight relative to the weight of the protein.

18. An MRI contrast medium which contains the nanoparticle of claim 1.

19. A drug delivery agent which contains the nanoparticle of claim 1.
